(19) United States
(12) Patent Application Publication
(10) Pub. No.: US 2009/0203794 A1
(43) Pub. Date: Aug. 13, 2009

(54) Aqueous Anaesthetic Composition Comprising Propofol

(76) Inventors: Gautam Vinod Daftary, Mumbai (IN); Srikanth Annappa Pai, Mumbai (IN); Mangesh Manikrao Kulkarnu, Thane (IN)

Correspondence Address: THORPE NORTH & WESTERN, LLP., P.O. Box 1219, SANDY, UT, 84091-1219, US

(21) Appl. No.: 11/991,203

(22) PCT Filed: Aug. 11, 2006
(86) PCT No.: PCT/IN2006/000299

§ 371 Date: Aug. 6, 2008

(30) Foreign Application Priority Data
Aug. 12, 2005 (IN) 940/MUM/2005

(51) Int. Cl. A61K 31/167; A61P 23/00

(52) U.S. Cl. 514/626

(57) ABSTRACT

The invention discloses an aqueous anaesthetic Propofol composition that is stable, autoclave sterilized, suitable for parental administration, having reduced incidence of pain upon injection. The composition comprises propofol, 2-hydroxypropyl-β-cyclodextrin and a local anaesthetic, Lignocaine.

### FIELD OF INVENTION

The present invention relates to aqueous anaesthetic compositions suitable for parenteral administration. In particular, it relates to aqueous solutions of Propofol complexed with 2-hydroxypropyl-β-cyclodextrin (referred to hereinafter as HPBCD).

### BACKGROUND AND PRIOR ART

Propofol is an intravenous anaesthetic agent used for the induction and maintenance of anaesthesia as well as sedation. It has the desirable property of causing a rapid induction of and quick recovery from anaesthesia.

Despite being a preferred anaesthetic agent, the aqueous insolubility of Propofol has presented significant difficulties. It was first formulated as an aqueous solution containing polyethoxylated castor oil as a solubiliser, but this was found to be unacceptable due to anaphylactoid reactions in some patients. Subsequently, Propofol was reformulated as an oil in water emulsion using a mixture of soya oil and purified egg phosphatide. However, these lipid-based emulsions still suffered from several limitations. In particular, the formulation causes pain on injection in a significant number of patients, especially when injected into a small vain. The emulsions are also particularly susceptible to microbial growth such that strict aseptic techniques must be maintained even where antimicrobial preservatives are used. The emulsions also show poor physical stability, the potential for embolism, and increased fat load.

These difficulties, and in particular the tendency of the Propofol emulsion to cause pain on injection, have resulted in a number of attempts to provide improved aqueous Propofol formulations. It is believed that it is the presence of Propofol in the aqueous phase is responsible for the pain on injection. A number of attempts have therefore been made to alter the pharmacokinetics and pharmacodynamics of the formulations used so as to shift more of the Propofol into the non-aqueous phase of the emulsion.

The possibility of adding a local anaesthetic to the emulsion so as to reduce the perceived pain on injection has also been investigated. However, the addition of local anesthetics or other electrolytes has been found to destabilize the Oil in water emulsion resulting in aggregation and coalescence of the oil phase. As such, premixed formulations containing oil in water Propofol emulsion and a local anaesthetic have proved to be pharmaceutically unacceptable. Where considered necessary, pre-treatment the area with a separate injection of local anaesthetic is therefore currently preferred.

As an alternative to using oil in water emulsions, several studies have investigated the possibility of using cyclodextrins or cyclodextrin derivatives to solubilise the Propofol. Cyclodextrins are cyclic compounds comprising a ring of six to eight glucopyranose units having a hydrophilic exterior surface and hydrophobic interior surface, and which act to solubilise hydrophobic compounds such as Propofol via inclusion complexation. Generally speaking, cyclodextrin derivatives are preferred due to the tendency of cyclodextrin to dissociate from the complexed hydrophobic compound on injection into the blood stream, as well as the tendency of cyclodextrin to interact preferably with cell membrane components.

One cyclodextrin derivative is 2-hydroxypropyl-β-cyclodextrin. G. Trapene at al (J. Pharm. Science, 1998, 84(4) (514 to 518)) discusses the physiochemical and anaesthetic properties of HPBCD:Propofol and indicates that a stable aqueous solution can be achieved with a 1:1 molar ratio of HPBCD:Propofol. International publication No. WO 96/32135 also discloses the use of Propofol: 2-hydroxypropyl-β-cyclodextrin complexes in which a Propofol to HPBCD molar ratio of 1:1.5 to 1:2.5 was shown to provide a stable aqueous solution of Propofol.

As described in Indian Patent No. 187686, the contents of which are incorporated herein by reference, the use of a propofol to HPBCD ratio from about 1:30 to about 1:60 results in an aqueous propofol formulation, which remains stable on autoclaving. There is, however, no provision for reduction in pain in this composition.

Another cyclodextrin derivative is sulfobutyl-ether-cyclodextrin (hereinafter referred to as SBECD). International publication No. WO 02/074200 discusses the use of SBECDs to solubilise Propofol. It also makes general reference to a list of local anesthetics to be mixed with SBECD: propofol solutions, although some of those suggested (eg. benzocaine, tetracaine) appear surprising in that they would result in systemic toxicity if intravenously injected.

However, the clinical usage of SBECD is yet to be established as safe, as no products containing SBECD are available commercially.

Thus there is a long standing need to have a stable single injectable composition of Propofol with a safe pain reducing local anaesthetic to facilitate easy one shot administration.

### OBJECT

The main object of the present invention is to provide a stable aqueous anaesthetic composition suitable for parenteral administration comprising propofol, 2-hydroxypropyl-β-cyclodextrin (HPBCD) and a safe local anaesthetic.

According to further aspect of the object of the present invention is to provide a suitable method for manufacturing an aqueous anaesthetic composition, suitable for parenteral administration, comprising Propofol, 2-hydroxypropyl-β-cyclodextrin (HPBCD), and a safe local anaesthetic.

### SUMMARY OF THE INVENTION

The present invention provides an aqueous anaesthetic composition suitable for parenteral administration comprising propofol, 2-hydroxypropyl-β-cyclodextrin (HPBCD) and a local anesthetic Lignocaine or its acid salts.

The method of manufacturing an aqueous anaesthetic composition suitable for parenteral administration, comprising forming an aqueous solution of propofol, 2-hydroxypropyl-β-cyclodextrin (HPBCD) and a local anaesthetic Lignocaine or its acid salts at pH 4-7 using buffers, and/or acids like hydrochloric acid, or phosphoric acids, and/or alkali like sodium hydroxide.

### DETAILED DESCRIPTION OF THE INVENTION

Presently, Propofol Injection and Lignocaine Injection are available separately. They are mixed just prior to administration. As the existing emulsion product is unstable on keeping after mixing with Lignocaine. The present invention now offers for the first time a single injection giving Propofol together with local anesthetic, Lignocaine to reduce pain on injection of Propofol.

Local anesthetics inhibit the conduction of sensory nerve impulses by permeating across the nerve cell membrane and reversibly complexing with the intra-cellular side of the sodium Ion pump, thereby decreasing the permeability of the nerve cell to sodium ions and thus inhibiting the transmission of nerve impulses. The chemical structure of a local anaesthetic comprises three groups, a lipophilic group (usually comprising a benzene ring) that allows the compound to permeate across the nerve cell membrane, an intermediate chain (usually comprising an ester or amide linkage), and an ionisable group (normally a tertiary amine) that allows the anaesthetic to be solubilised in the aqueous environment inside and outside the nerve cell.

The local anaesthetic may be used in its base form. It may react with phenolic (i.e. acidic) Propofol and hence acidic pH is useful in preventing such reaction and keeping the base free for reducing pain. The Lignocaine base may be used as an aqueous solution or in an acidic solution or as its water-soluble salts. It can also be used as a solution in carbonated base. Use of the local anaesthetic, Lignocaine in the form of its hydrochloride salt is generally preferred.

With this thinking, we have now found that Propofol, HPBCD and Lignocaine or its acid salts can be made to give stable aqueous compositions in acidic pH. An acidic pH additionally helps to control microbial growth during long term administration of the composition.

Preferably the pH of the composition is below 7, more preferably in the range of 4-7 and most preferably in the range 4.5 to 6.5.

The composition further comprises, acidifying agents and/or alkalising agents and/or antioxidants and/or buffers.

The antioxidant is selected from EDTA or a salt thereof, sodium metabisulphite, acetylcysteine, or ascorbic acid. Preferably the antioxidant is disodium edetate.

The compositions of the present invention may contain pharmaceutically acceptable acidifying agents and/or alkalizing agents and/or buffers for adjusting and stabilizing the pH of the solutions. Acidifying agents may include inorganic acids and/or organic acids and/or inorganic salts and/or organic salts. Alkalizing agents may include inorganic bases and/or organic bases and/or inorganic salts and/or organic salts. Examples of acidifying agents may be but are not limited to hydrochloric acid, carbonic acid, phosphoric acid, histidine HCl, glycine HCl, citric acid. Examples of alkalizing agents may be but are not limited to sodium hydroxide, potassium hydroxide, ammonium hydroxide, tromethamine, histidine.

Use of buffer helps in maintaining pH and increasing stability of the composition. The buffer may be selected from any pharmaceutically acceptable buffer systems such as, citrate buffer, phosphate buffer, histidine or glycine buffer containing any of the commonly used compounds, or a mixture of compounds such as citric acid. sodium citrate, potassium citrate, glycine, histidine, histidine HCl, phosphoric acid sodium phosphate, disodium hydrogen phosphate, sodium dihydrogen phosphate, potassium phosphate, dipotassium hydrogen phosphate. potassium dihydrogen phosphate.

The isotonic diluent may be selected from pharmaceutically acceptable diluents such as dextrose, sodium chloride and mannitol. However, glycerol or other polyols are not preferred.

Not bound by theory we believe that polyols such as glycerin, propylene glycol, or polyethylene glycol of a low molecular weight, act as co-solvents for propofol and increase free propofol in aqueous phase and increase pain on injection. Therefore use of such polyols is not preferred in these compositions.

The composition preferably has a Propofol:HPBCD weight ratio of 1:14-1:60. A weight ratio of propofol to HPBCD more preferably is 1:14-1:30 and most preferably 1:20-1:30.

The composition further comprises an antioxidant, a buffer, an isotonic diluent or a combination thereof.

Preferably, the propofol content of the solution in the present invention is 1 mg/ml-20 mg/ml and more preferably 1 mg/ml-10 mg/ml, most preferably about 10 mg/ml.

The local anaesthetic used in the composition of the present invention is Lignocaine base and/or its acid salts. Its content in the composition expressed as Lignocaine base is 0.5 mg/ml—1.5 mg/ml. More preferably the content of Lignocaine in the composition of present invention is about 1 mg/ml.

We have now found that aqueous compositions of the present invention comprising Propofol to HPBCD in the ratio of 1:14-1:60 and Lignocaine or its acid salts expressed as Lignocaine base 0.5-1.5 mg/ml of the composition at pH 4-7 are stable on autoclaving.

The composition of the present invention is manufactured by
1. *Dissolving HPBCD in required quantity of water or in a suitable buffer.
2. *Lignocaine as such or as a stock solution is added into BPBCD solution and dissolved by stirring and if required pH of the solution is adjusted between 4.5-7.0 with acidifying agents/alkalizing agents.
3. Propofol is added and dissolved by stirring under nitrogen cover.
4. The pharmaceutically acceptable additives like antioxidants, tonicity agents, as such or as stock solutions are added into Propofol—Lignocaine—BPBCD solution and mixed by stirring.
5. The resultant is then diluted to required volume and filtered through 0.45 and/or 0.22 μl filter.
6. The filtered solution is then filled into glass containers, head space of the containers are purged with Nitrogen gas before sealing and sterilized by autoclaving.

*Steps 1, and 2 may be done in alternative ways as follows:

Alternatively Lignocaine can be added as such or as a stock solution in acidic pH after complexing Propofol with HPBCD
OR

Lignocaine can be added into water acidic pH before the addition of HPBCD.

Thus in general, the method of manufacturing an aqueous anaesthetic composition suitable for parenteral administration, comprises forming an aqueous solution of propofol, 2-hydroxypropyl-β-cyclodextrin (HPBCD) and a local anaesthetic Lignocaine or its acid salts at pH 4-7 using buffers, and/or acids like hydrochloric acid or phosphoric acids, and/or alkali like sodium hydroxide.

### EXAMPLES

The invention will now be illustrated by way of Examples. The Examples are by way of illustration only and in no way restrict the scope of the invention.

Materials and Equipment Used in the Examples:

Propofol complies with the European Pharmacopoeia (Ph.Eur.) specifications,

Lignocaine hydrochloride (Lidocaine hydrochloride) complying with Indian Pharmacopoeia (I.P.) specifications is used.

Lignocaine (Lidocaine) complying with United States Pharmacopoeia (USP) specifications is used.

2-hydroxypropyl-β-cyclodextrin (HPBCD), Pharma grade is manufactured by M/s. Wacker Chemie.

### Example I

1% Propofol Lipid Free with 0.1% Lignocaine (as HCl)

The following composition was prepared by the procedure given below:

| Ingredients | Qty | |
|---|---|---|
| a) Propofol | 1 | g |
| b) 2-hydroxypropyl-β-cyclodextrin | 30 | g |
| c) Lignocaine HCl eq. to base | 0.1 | g |
| d) Disodium edetate | 0.0059 | g |

| | |
|---|---|
| e) Water for Injection | q.s. to 100 ml |

Procedure:

2-hydroxypropyl-β-cyclodextrin (30 g) was dissolved in 55 ml of Water for Injection. Lignocaine HCl (0.1234 g) was added into HPBCD solution and dissolved by stirring. Propofol was then added to HPBCD solution slowly under stirring. This solution was stirred at moderate speed for 3 hours to bring about complexation of Propofol with HPBCD.

Disodium edetate solution in water was added to the above solution under stirring. The volume was made up to 100 ml with water. The clear solution obtained was filtered through O.2μ filter, filled into glass vials under nitrogen, sealed and autoclaved.

The composition had a pH of 5.82

### Example II

Preclinical evaluation of Anesthetic Activity of composition of Example I. Induction and recovery time after administering anesthetic composition of Example I were evaluated in comparison with commercially available Propofol Oil-in-Water emulsion in Swiss albino mice.

The results are as given in Table 1:

TABLE 1

| Dose of Propofol | Induction time (seconds) | | Recovery time (minutes) | |
|---|---|---|---|---|
| (mg/ Kg body | | Propofol o/w | | Propofol o/w |
| weight) | Example I | emulsion | Example I | emulsion |
| 25 | 9.60 ± 0.97 | 9.90 ± 0.31 | 5.27 ± 2.23 | 4.29 ± 0.89 |

The results show that the Induction and recovery time of composition of Example I was comparable with that of Propofol Oil-in-Water emulsion indicating similar pharmacokinetic and pharmacodynamic profile of two pharmaceutically different compositions.

### Example III

### Acute Toxicity of Composition of Example I in Mice

The composition obtained in Example I was subjected to acute toxicity studies in mice. The composition of Example I was suitably diluted with 5% Dextrose Injection and administered intravenously. Propofol in the doses of 30 mg/kg, 35 mg/kg and 40 mg/kg body weight was administered in three different groups of animals, each group consisting of ten animals.

The animals were kept under observation for 14 days and mortality recorded at the end of 3 days and 7 days.

The mortality observed at different doses is provided in Table 2:

TABLE 2
Composition of Example I

| | |
|---|---|
| Dose | Mortality (%) |

| (mg/Kg body weight) | 3 days | 7 days |
|---|---|---|
| 35 | 20% | 20% |
| 40 | 60% | 60% |
| 45 | 80% | 80% |
| LD₅₀ | 39.23 | 39.23 |

### Example IV

### Stability Studies of Composition of Example I

The composition obtained in Example I was subjected to stability studies at 25° C. The stability data is provided in Table 3:

TABLE 3
Storage condition: 25° C.

| | Condition | | | |
|---|---|---|---|---|
| | | 25° C. | | |
| Test parameters | Initial | 1 month | 2 month | 3 month |
| Appearance | Clear, | Clear, | Clear, | Clear, |
| | colourless | colourless | colourless | colourless |
| | solution | solution | solution | solution |
| pH | 5.82 | 5.80 | 5.78 | 5.80 |
| *Assay of Propofol (%) | 100.1 | 99.0 | 99.80 | 98.50 |
| *Degradation products | | | | |
| Quinono Propofol | 0.03 | Nil | Nil | Nil |
| Propofol Dimer | 0.07 | 0.08 | 0.14 | 0.08 |
| *Assay of Lignocaine | 100.4 | 100.8 | 101.0 | 100.0 |
| HCl as Lignocaine (%) | | | | |

*Methods are given at the end of all Examples.

From the above it is evident that Propofol and Lignocaine (as HCl) are stable in the composition obtained in Example I without undergoing any degradation when stored at 25° C.

### Example V

### 1% Propofol Lipid Free with 0.1% Lignocaine (as Base)

The following composition was prepared by the procedure given below

| Ingredients | Qty | |
|---|---|---|
| a) Propofol | 1 | g |
| b) 2-hydroxyporpyl-β-cyclodextrin | 30 | g |
| c) Lignocaine base | 0.1 | g |
| d) Disodium edetate | 0.005 | g |
| e) Hydrochloric acid (0.1 N) | 2.0 | ml |

| | |
|---|---|
| f) Water For Injection | q.s. to 100 ml |

Procedure:

2-hydroxypropyl-β-cyclodextrin was dissolved in 55 ml of Water for Injection. Lignocaine base was added into HPBCD solution and dissolved by stirring. The pH of the solution was adjusted to about 6.7 with 0.1 N Hydrochloric acid. Propofol was then added to HPBCD solution slowly under stirring. This solution was stirred at moderate speed for 3 hours to bring about complexation of Propofol with HPBCD.

Disodium edetate was added to the above solution under, the volume was made up to 100 ml with water. The clear solution obtained was filtered through 0.2μ filter filled into glass vials under nitrogen, sealed and autoclaved.

The composition had a pH of 6.00.

Preclinical Evaluation of Anesthetic Activity of Composition of Example V

Induction and recovery time after administering anesthetic composition of Example V were evaluated in comparison with Propofol Oil-in-Water emulsion in Swiss albino mice.

The results are provided in Table 4:

TABLE 4

| Dose of Propofol | Induction time (seconds) | | Recovery time (minutes) | |
|---|---|---|---|---|
| (mg/Kg body | | Propofol o/w | | Propofol o/w |
| weight) | Example V | emulsion | Example V | emulsion |
| 25 | 9.90 ± 0.32 | 9.90 ± 0.31 | 4.61 ± 1.41 | 4.29 ± 0.89 |

The Induction and recovery time of composition of Example V was comparable with that of Propofol Oil-in-Water emulsion indicating similar pharmacokinetic and pharmacodynamic profile of two pharmaceutically different compositions

The composition of Example V was subjected for stability studies at 25° C. The data at the end of 3 months is provided in Table 5:

TABLE 5

| | Condition | | | |
|---|---|---|---|---|
| | | 25° C. | | |
| Test parameters | Initial | 1 month | 2 month | 3 month |
| Appearance | Clear, | Clear, | Clear, | Clear, |
| | colourless | colourless | colourless | colourless |
| | solution | solution | solution | solution |
| pH | 6.60 | 6.60 | 6.55 | 6.54 |
| *Assay of Propofol | 97.50 | 97.58 | 97.20 | 97.80 |
| (%) | | | | |
| *Degradation products | | | | |
| Quinono Propofol | 0.03 | Nil | Nil | Nil |
| Propofol Dimer | 0.06 | 0.07 | 0.01 | 0.053 |
| *Assay of Lignocaine | 101.2 | 101.8 | 102.0 | 100.9 |
| (%) | | | | |

*Methods are given at the end of all Examples.

From the above data it is evident that Propofol and Lignocaine (as base) are stable in the composition obtained in Example V without. undergoing any significant degradation when stored at 25° C.

Other compositions of the present invention are represented in Table 6, Table 7 and Table 8.

TABLE 6

| Ingredients | Example VI | |
|---|---|---|
| Propofol | 1.0 | g |
| Lignocaine base | 0.1 | g |
| HPBCD | 30.0 | g |
| Disodium edentate | 0.0055 | g |
| Disodium Hydrogen phosphate | 0.01 | g |
| Potassium dihydrogen phosphate | 0.0425 | g |
| Phosphoric acid 0.5% | 4 | ml |

| | |
|---|---|
| Water for Injection | q.s 100 ml |
| pH | 6.50 |

TABLE 7

| | Example | | Example | | Example | |
|---|---|---|---|---|---|---|
| Ingredients | VII | | VIII | | IX | |
| Propofol | 1.0 | g | 1.0 | g | 1.0 | g |
| Lignocaine base | — | | 0.1 | g | — | |
| Lignocaine HCl eq. to | 0.1 | g | — | | 0.1 | g |
| base | | | | | | |
| HPBCD | 22.0 | g | 22.0 | g | 14.0 | g |
| Disodium edetate | 0.0055 | g | 0.0055 | g | 0.0055 | g |

| | | | | |
|---|---|---|---|---|
| Hydrochloric acid 0.1N | — | 4.0 | ml | — |

| | | | | |
|---|---|---|---|---|
| Dextrose | — | — | 2.0 | g |

| | | | |
|---|---|---|---|
| Water for injection | q.s. 100 ml | q.s. 100 ml | q.s. 100 ml |
| pH | 6.0 | 5.94 | 6.0 |

TABLE 8

| | Example | | Example | | Example | | Example | |
|---|---|---|---|---|---|---|---|---|
| Ingredients | X | | XI | | XII | | XIII | |
| Propofol | 1.0 | g | 1.0 | g | 1.0 | g | 1.0 | g |
| Lignocaine base | — | | 0.1 | g | — | | 0.1 | g |
| Lignocaine HCl eq. to | 0.1 | g | — | | 0.1 | g | — | |
| base | | | | | | | | |
| HPBCD | 14.0 | g | 14.0 | g | 30.0 | g | 30.0 | g |

| | | | | | | |
|---|---|---|---|---|---|---|
| Disodium edetate | 0.0055 | g | 0.0055 | g | — | — |

| | | | | | | |
|---|---|---|---|---|---|---|
| Hydrochloric acid 0.1N | — | 4.0 | ml | — | 4.2 | ml |

| | | | | |
|---|---|---|---|---|
| Water for injection | q.s. 100 ml | q.s. 100 ml | q.s. 100 ml | q.s. 100 ml |
| pH | 6.0 | 5.94 | 5.98 | 6.02 |

The compositions shown in Table 6, Table 7 and Table 8 were prepared by the procedure given below.

### Example VI

The compositions were prepared by following the procedure of Example V using the components in the amounts mentioned in Table 6. Buffering salts and phosphoric acid whenever present were added as aqueous solution to BPBCD solution before adding lignocaine.

### Example VIII, XI & XIII

The compositions were prepared by following the procedure of Example V using the components in the amounts mentioned in Table 6 and Table 7.

### Example VII, X & XII

The compositions were prepared by following the procedure of Example I using the components in the amounts mentioned in Table 6 and Table 7.

### Example IX

The composition was prepared by following the procedure of Example I adding Dextrose at the end before filtration.

Method for Determination of Propofol Content, Degradation Products And Lignocaine Content:
1. Propofol and degradation products content: Propofol and degradation products content was determined by HPLC. The details are as follows:
   Column—Hypersil ODS
   Detector—Ultraviolet detector
   Detection wavelength—270 nm
   Mobile phase—60:15:25 Acetonitrile:methanol: 10 mM potassium phosphate Buffer
   Sample concentration—0.2 mg/ml
   Flow rate—1 ml/min.
2. Lignocaine Content:
   Column—Hypersil ODS
   Detector—Ultraviolet detector
   Detection wavelength—235 nm
   Mobile phase—60:15:25 Acetonitrile:methanol: 10 mM potassium phosphate Buffer
   Sample concentration—0.02 mg/ml
   Flow rate—1 ml/min.

### ADVANTAGES OF THE PRESENT INVENTION

The present invention provides a clear sterile anaesthetic composition that overcomes the disadvantages of emulsion formulation. The composition of the present invention has many advantages some of which are as follows:
1. The composition causes less pain on injection.
2. The composition is clear, can be visually inspected before administration and can be administered with the use of on-line microbial filter.
3. The composition does not contain phospholipids. Hence plasma phospholipids are unaffected on parenteral administration of the composition.
4. The composition does not cause any change in triglyceride clearance.
5. The composition can be mixed with any of the commonly used diluents before administration.
6. The acidic pH of the composition helps to control microbial growth during long term administration.

1. An aqueous anaesthetic composition suitable for parenteral administration comprising propofol, 2-hydroxypropyl-βcyclodextrin (HPBCD) and a local anesthetic Lignocaine or its acid salts.

2. A composition according to claim 1, wherein the Lignocaine acid salt is Lignocaine hydrochloride.

3. A composition as claimed in claim 1, wherein the said composition has a pH below 7.

4. A composition as claimed in claim 3, wherein the said composition has a pH 4-7.

5. A composition as claimed in claim 1, further comprising an antioxidant, a buffer, an isotonic diluent or a combination thereof.

6. A composition as claimed in claim 1, wherein the weight ratio of propofol to HPBCD is from 1:14-1:60.

7. A composition as claimed in claim 6, wherein the weight ratio of propofol to HPBCD is from 1:14-1:30.

8. A composition as claimed in claim 7, wherein the weight ratio of propofol to HPBCD is from 1:20-1:30.

9. A composition as claimed in claim 1, wherein the propofol content of the composition is from 1 mg/ml-20 mg/ml.

10. A composition as claimed in claim 9, wherein the propofol content of the composition is about 10 mg/ml.

11. A composition as claimed in claim 1, wherein the Lignocaine or its acid salts content of the composition expressed as Lignocaine base is from 0.5 mg/ml-1.5 mg/ml.

12. A composition as claimed in claim 11, wherein the Lignocaine or its acid salts content of the composition expressed as Lignocaine base is about 1 mg/ml.

13. A composition as claimed in claim 1, wherein Propofol is about 1 g; 2-hydroxypropyl-β-cyclodextrin is about 30 g; Lignocaine HCl eq. to base, is about 0.1 g; Disodium edetate is about 0.006 g and Water for Injection q.s. to 100 ml and has a pH 4-7.

14. A composition as claimed in claim 1, wherein Propofol is about 1 g; 2-hydroxypropyl-β-cyclodextrin is about 30 g; Lignocaine base is about 0.1 g; Disodium edetate is about 0.005 g; Hydrochloric acid 0.1N is about 2 ml and Water for Injection q.s. to 100 ml and has a pH 4-7.

15. A method of manufacturing an aqueous anaesthetic composition as claimed in claim 1 which is suitable for parenteral administration, comprising forming an aqueous solution of propofol, 2-hydroxypropyl-β-cyclodextrin (HPBCD) and a local anaesthetic Lignocaine or its acid salts at pH 4-7 using buffers, and/or acids like hydrochloric acid, or phosphoric acids, and/or alkali like sodium hydroxide.

16-17. (canceled)
